(19) United States
(12) Patent Application Publication
(10) Pub. No.: US 2024/0188836 A1
(43) Pub. Date: Jun. 13, 2024

(54) Integrated Sensing Device for Heart Sound and ECG Signals

(71) Applicant: Decentralized Biotechnology Intelligence Co., Ltd., Taipei (TW)

(72) Inventors: Yao-Sheng Chou, Taipei (TW); Wei-Sheng Su, Taipei (TW); Hsiao-Yi Lin, Taipei City (TW)

(21) Appl. No.: 18/507,062

(22) Filed: Nov. 12, 2023

(30) Foreign Application Priority Data
Dec. 8, 2022 (TW) 111147273

(51) Int. Cl. A61B 5/0205; A61B 5/00; A61B 5/33; A61B 7/04; G16H 40/63; G16H 50/20

(52) U.S. Cl. CPC A61B 5/0205; A61B 5/33; A61B 5/7203; A61B 7/04; G16H 40/63; G16H 50/20; A61B 8/0883

(57) ABSTRACT

An integrated sensing device for heart sounds and electrocardiographic signals, which includes a plurality of integrated sensing units for heart sounds and electrocardiographic signals, each sensing unit having a flexible base with upper and lower surfaces, and a plurality of electrodes been arranged on the lower surface of the flexible base, a piezoelectric layer arranged on the upper surface of the flexible base, a piezoelectric electrode formed on the piezoelectric layer, a metal buckle passing through the flexible base and the piezoelectric layer, as an electrical connection to the plurality of electrodes. The pluralities of electrodes are used for sensing body electrocardiogram signals, and one of the plurality of electrodes together with the piezoelectric electrode are used for sensing body heart sound signals. A system circuit board is used to filter, to amplify, and to digitalize collected multiple heart sounds and multiple ECG signals.

### CROSS-REFERENCE STATEMENT

The present application is based on, and claims priority from, TAIWAN Patent Application Serial Number 111147273, filed Dec. 8, 2022, the disclosure of which is hereby incorporated by reference herein in its entirety.

### BACKGROUND

### Technical Field

The present invention relates to a sensing device, more specifically, an integrated sensing device for heart sound and ECG signals.

### Related Art

The circulatory, respiratory and gastrointestinal systems are the major and primary body systems which may provide critical information for medical purposes. The most well-known medical monitoring measure is called auscultation which is a kind of medical technique for collecting sound signal by a stethoscope, followed by transmitting the sound signal to the doctor's ears through a pair of hollow tubes.

For the cardiovascular inspection, the cardiac auscultation is a fairly rapid and basic diagnostic method.

ECG and heart sound signals are physiological signals generated by the heart contractions, they are important factors and means for diagnosing heart diseases. The ECG signal is the index of the physiological activity of the heart, while the heart sound signal is the activity presentation of the heart and the cardiovascular system.

The heart sound signal includes physiological message and pathological information of the heart. The heart sound mainly includes the first heart sound and the second heart sound. It is difficult to distinguish the first heart sound from the second heart sound under the interference of external noise and heart murmurs. Therefore, it is also an essential task to determine the heart murmur and the arrhythmia issues, simultaneously.

The ECG signal is typically employed to detect general heart diseases, such as myocardial infarction and arrhythmia, in clinical diagnosis. However, it is unlikely to reflect some symptoms caused by abnormal heart tissue, it relies on the heart sound signal inspection.

Moreover, the single electrocardiogram information is unlikely to determine whether cardiac arrest or not, when pulseless electrical activity (PEA) situation occurs in clinical cases. Therefore, how to make a diagnosis based on the phonocardiogram with the ECG is very important.

In view of this, what is required is to provide a wearable heart rate sensing device that integrates heart sound detection for improving the efficiency of clinical and health care inspections.

### SUMMARY

Based on above, the purpose of the present invention is to provide an integrated sensing device for heart sounds and ECG signals.

In one aspect, the integrated sensing device includes sensing units to capture heart sounds and ECG signals at multiple points corresponding to different positions of the body; each sensing unit includes a flexible substrate having an upper surface and a lower surface, and a bottom electrode is arranged on the lower surface of the flexible substrate. A system circuit board is electrically connected to the integrated sensing unit for performing filtering, amplification and pre-processing on the collected heart sounds and ECG signals. A wireless transmission module, which transmits the background noise-free and stable pre-processed heart sound signals and ECG signals to an external computing device coupled with the integrated sensing device for further analysis.

In another aspect, the flexible substrate includes a fabric, polysiamine (PI) or polyethylene terephthalate (PET).

The piezoelectric layer includes polyvinylidene fluoride (PCDF) or lead zirconate titanate (PZT).

In one aspect, the system circuit board is electrically connected to the sensing units for preprocessing collected heart sounds and ECG signals. The system circuit board includes a signal pre-processing module for filtering, amplifying and digitalizing the heart sound and ECG signals to generate preprocessing signals. A microprocessor is electrically connected to the signal pre-processing module to receive the pre-processing signal to obtain heart murmur free signals, and thereafter, to store the noise free signals in a storage unit. An external computing device analyzes and cross-compares the heart murmur free signals. Steps of the analysis and cross-comparison include signal processing, feature extraction, feature point comparison, and classification.

A piezoelectric layer is arranged on the upper surface of the flexible substrate. A piezoelectric electrode is disposed on the piezoelectric layer, and a metal buckle passes through the flexible substrate and the piezoelectric layer.

In one embodiment, the above-mentioned external computing device is a smart phone or a cloud server.

In one embodiment, the background noise-removed and stable pre-processed heart sound and ECG signals are analyzed and cross-compared by the electronic computing device.

In one embodiment, the external computing device may fetch the pre-processed and digitized multi-channel (multi-dimensional) ECG and heart sound data. The present invention analyzes and compares the collected/received pre-processing and digitized multi-channel (multi-dimensional) signals, the further analysis and comparison includes, for example, signal processing (including wavelet analysis, Fourier transform, etc.), feature extraction, artificial intelligence (artificial intelligence; AI) comparison of feature points to determine whether the patient is abnormal or not. Subsequently, the results of the comparison and status classification are sent to the database for subsequent comparison references, and the data is evaluated by the medical institutions for medical advice.

In one embodiment, the artificial intelligence (AI) comparison and status classification is performed through the AI algorithm installed in the external computing device.

In one aspect, the classification is used to classify normal and abnormal heart sounds and ECG signals by AI algorithms which perform the following steps by an electronic computing equipment: pre-filtering and normalizing input heart sounds and ECG signals; extracting time-domain and frequency-domain features from the pre-filtered and normalized heart sounds and electrocardiograms; outputting classification results by Convolutional Neural Network (CNN) model.

### BRIEF DESCRIPTION OF THE DRAWINGS

FIG. 1A shows a side view of an integrated heart sound and ECG sensing device according to an embodiment of the present invention.

FIG. 1B and FIG. 1C respectively show the top view and bottom view of the integrated heart sound and ECG sensing device according to an embodiment of the present invention.

FIG. 1D shows a bottom view of the integrated heart sound and ECG sensing device according to another embodiment of the present invention.

FIG. 2 shows the configuration of the integrated heart sound and ECG sensing device according to an embodiment of the present invention.

FIG. 3 shows a functional block diagram according to an embodiment of the present invention.

FIG. 4 shows the waveforms of heart sound signals and ECG signals according to an embodiment of the present invention.

FIG. 5 shows a system diagram according to an embodiment of the present invention.

FIG. 6 shows an example of a processing system running on a mobile device or a cloud server according to an embodiment of the present invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

The present invention provides a heart rate detector with a wireless electronic stethoscope for diagnosis during auscultation. The doctor may observe the heart rate/heart sound signal displayed on the mobile device, simultaneously. By visualizing the heart rate/heart sound signals with the normal diagnosis procedure, the present invention offers real time arrhythmia and heart murmur inspection at the same time for effectively improving the clinical and health care inspections.

Conventionally, the electrocardiograph device or the stethoscope is typically means used by the cardiologists to fetch the heart beats and sound signal for preliminary diagnosis. However, the conventional device is unable to simultaneously analyze/measure ECG, heart sound signals due to the equipment limitation.

The systolic period is the period of contraction of the ventricles of the heart that occurs between the first and second heart sounds of the cardiac cycle (the sequence of events in a single heart beat). Systole causes the ejection of blood into the aorta and pulmonary trunk. In the present invention, the heart sound analysis is synced with the ECG analysis. Therefore, the present invention provides complete systolic period and abnormal heart sound detecting results to assist the doctors for medical inspection at critical moments.

The traditional heart sound device collets signal based on a heart sound probe or conventional microphone that is not only relatively large but also difficult to implement within the wearable applications.

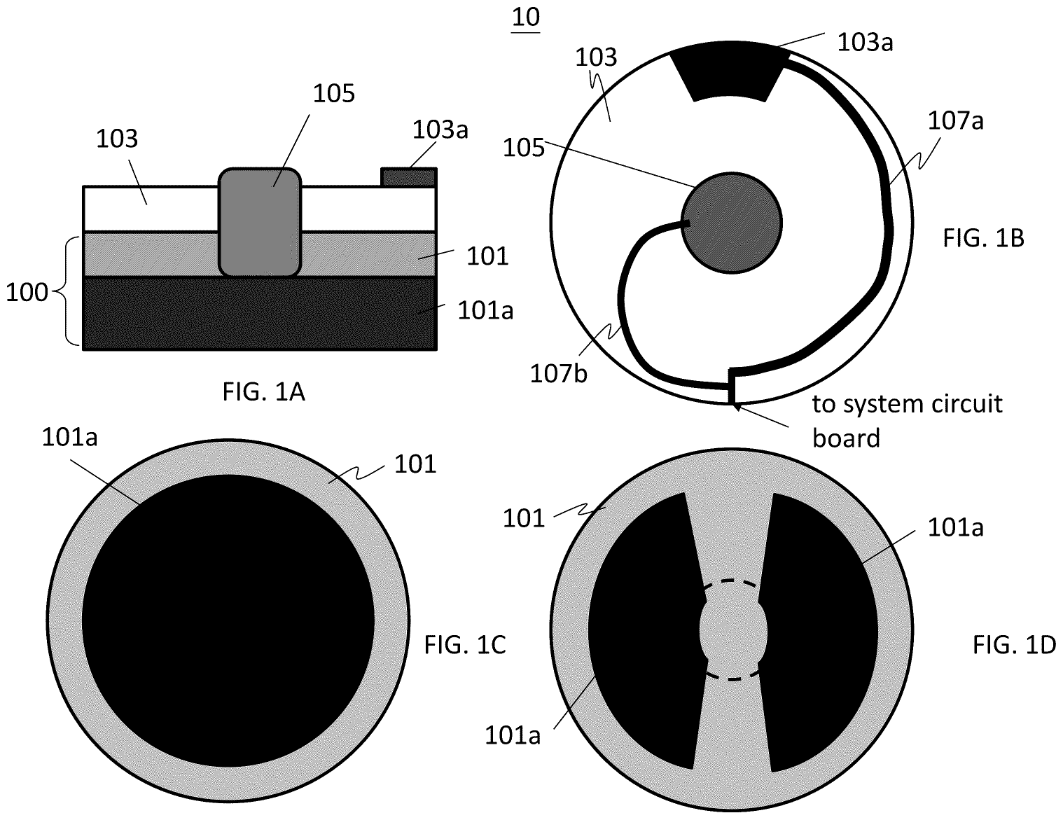

FIG. 1A-FIG. 1D show an integrated sensing device 10 for heart sound and electrocardiogram detection according to an embodiment of the present invention. The sensing device 10 integrates piezoelectric materials onto a substrate 100 with electrodes.

The integrated heart sound and ECG sensing device 10 includes a substrate 100 having an insulating fabric 101 which has electrodes 101a formed thereon. A piezoelectric material layer 103 (for example, polyvinylidene fluoride (PCDF), lead zirconate titanate (PZT) or the like) and a metal buckle 105 is provided. The metal buckle 105 penetrates through the insulating fabric 101 and the piezoelectric material layer 103. The bottom electrode 101a is on the insulating fabric 101 (single electrode as shown in FIG. 1C, and multiple electrodes as shown in FIG. 1D). Further, the surface of the piezoelectric material layer 103 is plated with at least one piezoelectric electrode 103a for cooperating with the single bottom electrode 101a or multiple bottom electrodes 101a to collect vibration signals (for detecting sound signals).

FIG. 1A shows a side view of the integrated sensing device 10 for heart sound and ECG detection. FIG. 1B shows a top view of the same integrated sensing device 10, wherein the piezoelectric electrodes 103a are electrically connected to the system circuit board (not shown) through wires 107a connection. The metal buckle 105 is electrically connected to the system circuit board through the wire 107b. FIG. 1C shows a bottom view of the integrated sensing device 10 with single bottom electrode 101a disposed on the insulating fabric 101. In addition to the arrangement of a single electrode, the multiple bottom electrodes maybe formed on the insulating fabric 101 as shown in FIG. 1D according to the actual practice.

In addition to textiles, the aforementioned substrate 100 may be made of plastics such as polysiamine (PI), polyethylene terephthalate (PET) or the like.

In one embodiment, the integrated heart sound and ECG sensing device 10 is fabricated with a patch form.

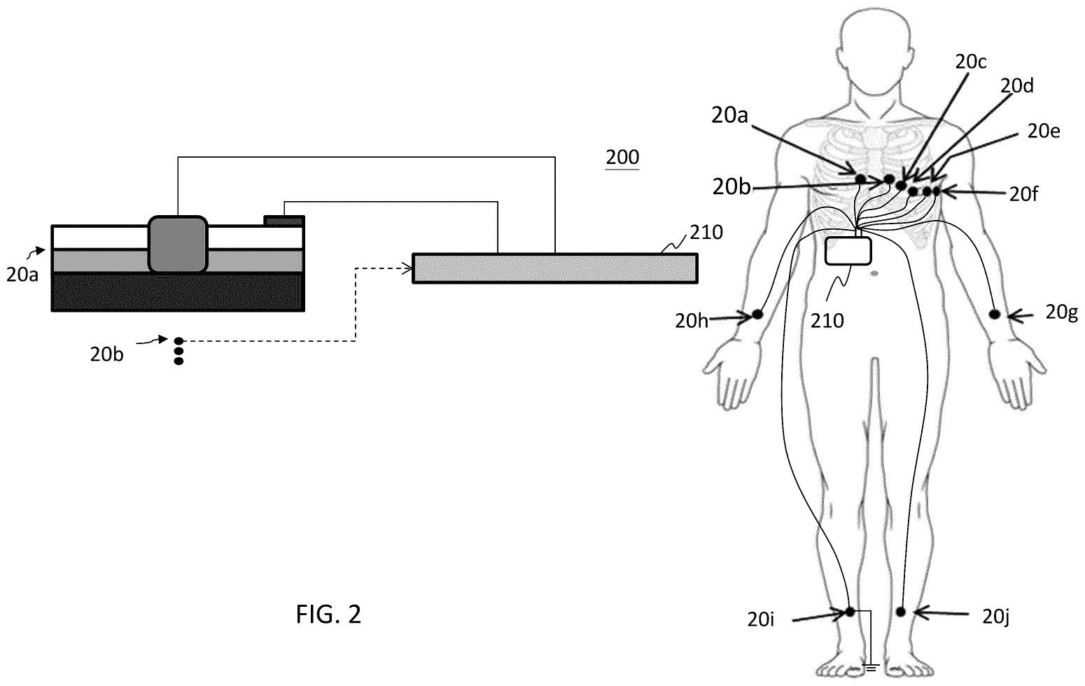

ECG and heart sound signals are employed to measure heart activity from different aspects, much more comprehensive indexes can be obtained by multiple parameter analysis. Therefore, the present invention provides the integrated heart sound and ECG sensing device 200, the configuration of which is shown in FIG. 2. The integrated sensing device 200 includes multiple integrated sensing units (20a, 20b, 20c, . . . ) that are electrically connected to the system circuit board 210. In the integrated sensing device 200 for heart sounds and ECG, the integrated sensing units (20a, 20b, 20c, . . . ) are separately electrically connected to the system circuit board 210 (as shown in FIG. 2), or electrically connected with the system circuit board 210 in an array manner to form a single wearable device for simultaneously detecting the ECG and heart sound signals of the body (such as human body) 30 by multi-points manner. The multiple heart sound and ECG integrated sensing units (20a, 20b, 20c, . . . , 20i, 20j) are respectively placed at the position corresponding to usual ECG detecting positions (V1, V2, V3 . . . , V6, RA, LA, RL, LL). For example, V1 is located in the fourth intercostal space on the right border of the sternum, V2 is located in the fourth intercostal space on the left border of the sternum, V3 is located in the middle of V2 and V3, and V4 is located in the fifth intercostal space in the middle of the left clavicle.

In one embodiment, the pluralities of heart sound and ECG integrated sensing units (20a, 20b, 20c, . . . ) are electrically connected to the system circuit board 210 through wire connection or wireless connection to transmit signal.

In one embodiment, the system circuit board 210 includes a signal preprocessing circuit, an analog-to-digital conversion circuit, a memory, a processor, a charging circuit, a power management circuit, a wireless transmission module, and the like.

In one embodiment, the system circuit board 210 is integrated and packaged into a flexible substrate to form a flexible circuit board.

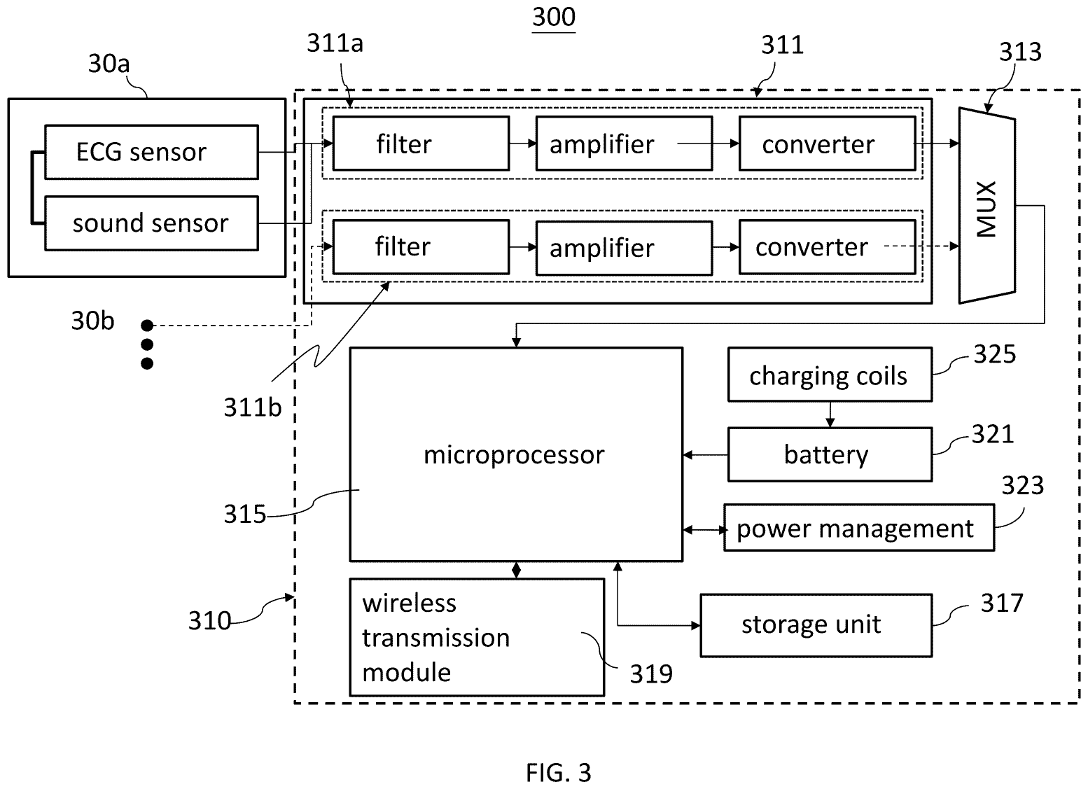

FIG. 3 is a functional block diagram of the integrated sensing device 300 for heart sound and ECG signals, wherein each individual unit (for example 30a) in the multiple integrated sensing units (30a, 30b, . . . ) may detect the electrocardiographic signal and heart sound from the body. The detected electrocardiographic signal and the heart sound are processed with filtering, signal amplification, and analog/digital conversion by a pre-process channel 311a in a pre-processing circuit 311 to obtain pre-processed digitized ECG and sound signal. Similarly, other signal detected by another heart sound and ECG integrated sensing units (such as 30b) is also filtered, amplified, and converted through another corresponding signal pre-processing channel 311b to obtain another set of pre-processed digitized ECG signal and sound signal.

In one embodiment, each pre-processing channel includes a filter, a signal amplifier, and an analog/digital (A/D) converter.

Because the signals captured by different integrated sensing units are correspond to different detected signals of different body parts, therefore, they have time correlation. The digitized multi-channel ECG and heat sound signals processed by the processing channels (such as 311a, 311b) are multiplexed by a multiplexer (MUX) 313, and then fed into the microprocessor 315 for further computing and processing, Finally, the stable ECG and sound signals are obtained without background noise.

The microprocessor 315 stores the stable and noise-free ECG and sound signal in the storage unit 317 by instructions or programs, or the signals is sent to an external mobile device through the wireless transmission module 319 for further analysis. The aforementioned mobile device may be a smart phone, a tablet computer or a cloud server.

The battery pack 321 provides power for the integrated sensing device 300 and it cooperates with the power management module 323 to optimize power usage. Battery pack 321 may also be wirelessly charged by charging coils 325.

In one embodiment, the single system board 310 may integrate the components such as the microprocessor 315, the storage unit 317, the wireless transmission module, the signal preprocessing circuit 311, the MUX 313 and the power management module 321.

In one embodiment, the user can check, analyze and compare the collected electrocardiogram (ECG) and phonocardiogram (PCG) through an external computing device. The external computing device may be a smart phone, a tablet computer or a cloud server.

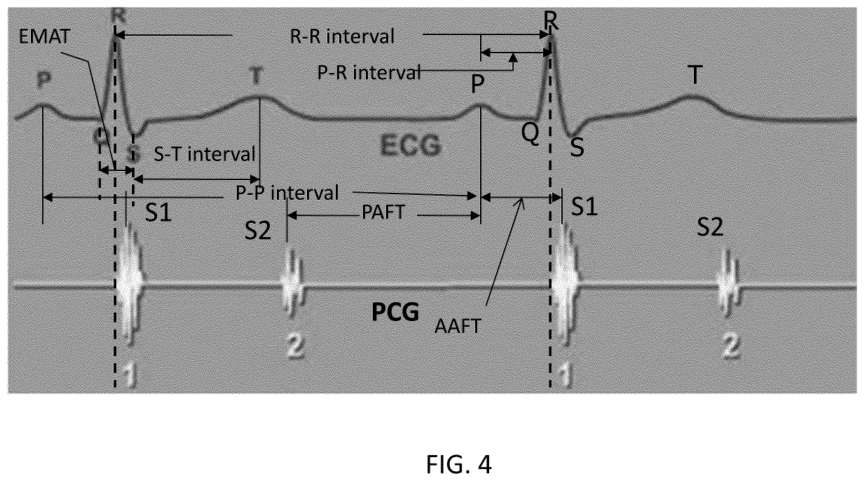

In one embodiment, the heart sounds/ECG waveforms are displayed by the external computing device, as shown in FIG. 4. The external computing devices displays the multi-channel electrocardiogram (ECG) and phonocardiogram (PCG). The phonocardiogram noise reflects the heart sound murmur, and the relative position between the two waveforms reflects the dynamic characteristics of the heart.

The electrocardiogram indexes include: P-P interval, P-R interval, and S-T interval.

Electrocardiogram and phonocardiogram comprehensive indexes: EMAT (time interval from the starting point of Q wave to the maximum amplitude point of S1), AAFT (time interval from the starting point of P wave to S1), PAFT (time interval from S2 to P wave).

EMAT is related to heart failure evaluation index; PAFT and AAFT are related to acute myocardial infarction and myocardial ischemia evaluation indexes.

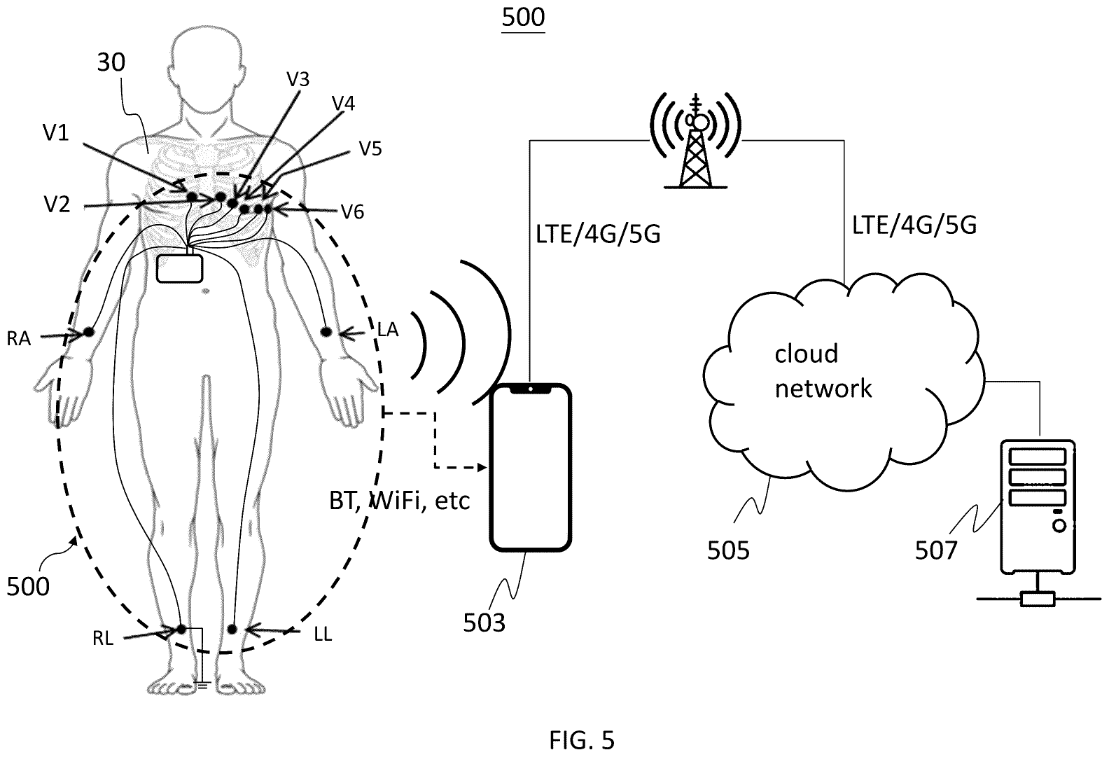

As shown in FIG. 5, the integrated sensing device 500 for heart sound and ECG signals includes pluralities of integrated sensing units and the system circuit board 310 (refer to FIGS. 2-3), it is worn on the user 30 and coupled with the external computing device (for example, the smart phone, the tablet computer) 503. The heart sound data collected by the integrated heart sound and ECG sensing device 500 can be uploaded to the cloud server by the mobile device 503 via the cloud network 505 through wireless transmission (for example, wireless communication such as Bluetooth and WiFi) 507. In the cloud server, the data will be stored in the cloud database. The above system also includes an application program installed in the mobile device, the application program includes instructions for receiving and sending data between the integrated sensing device 500, the mobile device 503 and the cloud server 505. The aforementioned application program is operated based on the Android, Windows 10 or iOS operating system platform, and can upload relevant collected data/signals, such as ECG signals, heart sound signals and their waveforms, to the cloud server 505 for storage, and the signals are provided for further data analysis and feature extraction by algorithms to generate an evaluation report for medical advice.

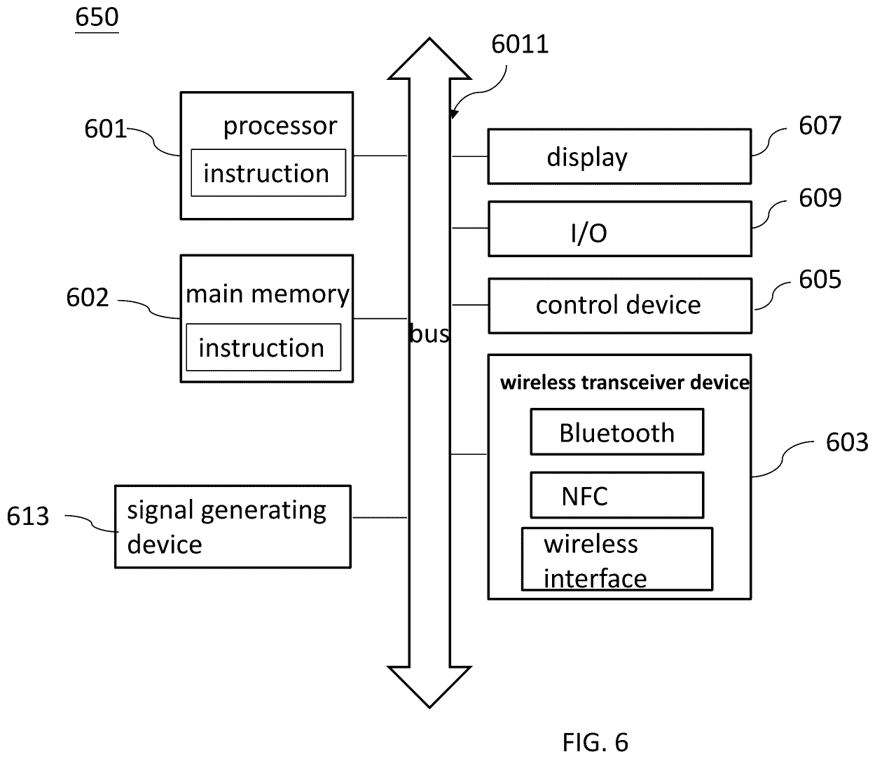

FIG. 6 shows a functional block diagram of the processing system 650 of the mobile device 503 or computing systems of the cloud server 507 (FIG. 5) according to the present invention. The cloud server 507 (or mobile device) executes instructions to perform computing processes, such as performing the previously described algorithms for detecting cardiac abnormalities and extracting ECG signal features. Those skilled in the art should understand that the above instructions can be stored and/or executed by hardware, software or firmware without departing from the spirit of the present invention. Furthermore, those skilled in the art will appreciate that the exact configuration of each processing system may be modified, and the processing system 650 shown in FIG. 6 is merely an example.

The processing system 650 includes a processor 601, a main memory 602, a wireless transceiver device 603 (including a Bluetooth module, a near field communication (NFC) module, etc., and a wireless network interface), a control device 605 (such as a keyboard and pointer device), a video display 607, an input/output (I/O) device 609, and a signal generating device 613 coupled with a bus 6011.

The main memory 602, the wireless transceiver device 603, the video display 607, the input/output (I/O) device 609, and any number of other peripheral devices are connected to the microprocessor 601 through the bus (BUS) 6011 to exchange data with the processor 601 for application programs executed by the processor.

The wireless transceiver device 603 is connected to the antenna to transmit and receive voice and data signals through the wireless communication channel. In one embodiment, the wireless communication channel may be a digital wireless communication channel, such as WiFi, Bluetooth, RFID, NFC, 3G/4G/5G or any other future wireless communication interface.

The video display 607 receives data from the processor 601 and displays images on the screen for users. The video display 607 may be a liquid crystal display (LCD) or an organic light emitting diode (OLED) display.

The main memory 602 is a device for storing the data, and sending/receiving data to/from the processor 601. The main memory 602 may include non-volatile memory, such as read only memory (ROM), which stores instructions and data for operating subsystems of the processing system. Those skilled in the art will appreciate that any number of memories may be used to perform this function. Main memory 602 may also include volatile memory, such as random-access memory (RAM), which stores instructions and data required by the processor 601 to perform computing, such as the processing required by the system according to the present invention. Those skilled in the art will understand that any type of memory can be used herein, and the type of actual usage is a design choice in the art.

The Bluetooth module allows the processing system 650 to establish communication with similar devices such as the wearable stethoscope device 101 based on the Bluetooth standard. The near field communication (NFC) module allows the integrated sensing device 500 to establish wireless communication with another similar device within the near field range. Other peripheral devices that are connected to processor 601 include a Global Positioning System (GPS) and other positioning transceivers.

The processor 601 includes a microprocessor, or any combination of a processor, which executes the processing, instructions according to the present invention. The processor 601 executes various application, programs stored in the storage unit. These applications or programs receive user input via a display with a touch screen or directly from the keyboard. Some applications in the main memory 602 is executable by the processor 601, and they include the applications developed by UNIX, Android, iOS, Windows, or other platforms.

In the integrated sensing device according to the present invention, each sensing unit is formed by integrating the piezoelectric film with the fabric having electrocardiographic electrodes (refer to FIGS. 1-2), since the detected sound signal includes heart, lung sound signal, etc. These signals are collected by the piezoelectric film, and the vibration signal is transmitted by the fabric attached to the body, the sound sensing device eliminates the conventional microphone structure, thereby achieving simpler, thinner sensors, and the design is very convenient for the users to wear on the body. The integrated sensing device offers simultaneous inspection and multiple points detection to capture the ECG and heart sound signals. It also provides cross-comparison between ECG and heart sound signals at different positions to achieve more accurate results for early detection of cardiac abnormalities.

In one embodiment, the above-mentioned external computing device, for instant, a smart phone or a server, can obtain the pre-processed and digitized multi-channel (multi-dimensional) ECG and heart sound data detected by the integrated sensing device through a wireless transmission interface. The heart sounds and ECG is viewable to monitor the ECG and PCG waveforms stored in non-volatile memory in real time, and followed by transmitting the heart sounds and ECG waveforms to the cloud data center for data storage.

In one embodiment, the aforementioned external computing device, such as a smart phone or a cloud server, may fetch the pre-processed and digitized multi-channel (multi-dimensional) ECG and heart sound data detected by the integrated sensing device through a wireless transmission interface. The present invention offers further analysis and comparison of collected/received pre-processing and digitized multi-channel (multi-dimensional) signals, the further analysis and comparison includes, for example, signal processing (including wavelet analysis, Fourier transform, etc.), feature extraction, artificial intelligence (artificial intelligence; AI) comparison of feature points with previous data in a database (such as a big data database), and condition classification to determine whether the patient is abnormal or not. Subsequently, the results of the comparison and status classification are sent to the database for subsequent comparison references, and the data is evaluated by the medical institutions for medical advice.

In one embodiment, the aforementioned database may be a cloud database coupled to a cloud server.

In one embodiment, the comparisons and status classifications are processed by AI algorithm installed in the external computing device to classify the normal or abnormal heart sound and ECG signals. The AI algorithm may perform the following steps: pre-filtering and normalizing the input heart sounds and ECG signals, extracting time domain and frequency domain features, using Convolutional Neural Network (CNN) models or other types of neural networks Network models (such as RNN/LSTM, etc.) output classification results, wherein the RNN refers to recurrent neural network model; LSTM refers to long-short-term memory model.

In addition to recording the electrocardiogram, the present invention is capable to identify the ventricle related normal or abnormal heart sounds, and monitor the electrocardiogram, heart sound, and systolic functions, followed by recording the diastole and systole parameters, and analyzing the diastole/systolic functions. Therefore, the present invention is beneficial for early screen out heart diseases, for example, arrhythmia and heart failure.

As will be understood by persons skilled in the art, the foregoing preferred embodiment of the present invention illustrates the present invention rather than limiting the present invention. Having described the invention in connection with a preferred embodiment, modifications will be suggested to those skilled in the art. Thus, the invention is not to be limited to this embodiment, but rather the invention is intended to cover various modifications and similar arrangements included within the spirit and scope of the appended claims, the scope of which should be accorded the broadest interpretation, thereby encompassing all such modifications and similar structures. While the preferred embodiment of the invention has been illustrated and described, it will be appreciated that various changes can be made without departing from the spirit and scope of the invention.

What is claimed is:

1. An integrated sensing device for heart sounds and ECG signals, comprising:
sensing units configured to capture multiple heart sounds and ECG signals at multiple different positions; each sensing units including a flexible substrate having an upper surface and a lower surface, and a bottom electrode on said lower surface of said flexible substrate;
a piezoelectric layer arranged on said upper surface of said flexible substrate;
a piezoelectric electrode disposed on said piezoelectric layer; and
a buckle passing through said flexible substrate and said piezoelectric layer.

2. The device of claim 1, wherein said bottom electrode is employed for sensing electrocardiogram signals, and said bottom electrode and said piezoelectric electrode being used for sensing heart sound signals.

3. The device of claim 2, further comprising a system circuit board electrically connected to said sensing units for pre-processing collected heart sounds and ECG signals.

4. The device of claim 3, wherein said system circuit board includes:
a signal pre-processing module for filtering, amplifying and digitizing said heart sound and ECG signals to generate preprocessing signals; a microprocessor electrically connected to said signal pre-processing module to receive said pre-processing signal to obtain noise free signals, and to store said noise free signals in a storage unit.

5. The device of claim 4, further including transmitting said noise free signals to an external computing device.

6. The device of claim 5, wherein said external computing device analyzes said noise free signals.

7. The device of claim 6, wherein said external computing device cross-compares said noise free signals.

8. The device of claim 7, wherein steps of said analysis and cross-comparison include:
signal processing, feature extraction, feature point comparison and classification.

9. The device of claim 8, wherein said classification is to classify normal and abnormal heart sounds and ECG signals by AI algorithms.

10. The device of claim 9, wherein said AI algorithms perform following steps by an electronic computing equipment:
pre-filtering and normalizing input heart sounds and ECG signals;
extracting time-domain and frequency-domain features from said pre-filtered and normalized heart sounds and electrocardiograms; and
outputting classification results for said time domain and frequency domain features by Convolutional Neural Network (CNN) model.

11. The device of claim 3, wherein said system circuit board is arranged on said flexible substrate.

12. The device of claim 1, wherein said flexible substrate includes a fabric, polysiamine (PI) or polyethylene terephthalate (PET).

13. The device of claim 1, wherein said piezoelectric layer includes polyvinylidene fluoride (PCDF) or lead zirconate titanate (PZT).

14. An integrated sensing device for heart sounds and ECG signals, comprising:
sensing units configured to capture multiple heart sounds and ECG signals at multiple different positions; each sensing unit including a flexible substrate having an upper surface and a lower surface, and a bottom electrode arranged on said lower surface of said flexible substrate for sensing electrocardiogram signals, and said bottom electrode and said piezoelectric electrode are used for sensing heart sound signals;
a piezoelectric layer arranged on said upper surface of said flexible substrate;
a piezoelectric electrode disposed on said piezoelectric layer;
a buckle passing through said flexible substrate and said piezoelectric layer;
a system circuit board electrically connected to said sensing unit for preprocessing collected heart sounds and ECG signals.

15. The device of claim 14, wherein said system circuit board includes:
a signal pre-processing module for filtering, amplifying and digitizing said heart sound and ECG signals to generate preprocessing signals; a microprocessor electrically connected to said signal pre-processing module to receive said pre-processing signal to obtain noise free signals, and to store said noise free signals in a storage unit.

16. The device of claim 15, further including transmitting said noise free signals to an external computing device to analyze and cross-compare said noise free signals.

17. The device of claim 16, wherein steps of said analysis and cross-comparison include:
signal processing, feature extraction, feature point comparison and classification.

18. The device of claim 17, wherein said classification is to classify normal and abnormal heart sounds and ECG signals by AI algorithms.

19. The device of claim 18, wherein AI algorithms perform following steps by an electronic computing equipment:
pre-filtering and normalizing input heart sounds and ECG signals;
extracting time-domain and frequency-domain features from said pre-filtered and normalized heart sounds and electrocardiograms; and
outputting classification results for said time domain and frequency domain features by Convolutional Neural Network (CNN) model.

20. The device of claim 14, wherein said piezoelectric layer includes polyvinylidene fluoride (PCDF) or lead zirconate titanate (PZT).
